(19) United States
(12) Patent Application Publication
(10) Pub. No.: US 2011/0038954 A1
(43) Pub. Date: Feb. 17, 2011

(54) CALCIUM-RICH ALKALINE POWER PRODUCED FROM PROCESSED MOLLUSC SHELLS

(76) Inventors: Hu Meng Bu, Victoria (AU); Xiao Ling Bu, Victoria (AU)

Correspondence Address: VEDDER PRICE P.C., 222 N. LASALLE STREET, CHICAGO, IL, 60601, US

(21) Appl. No.: 12/740,928

(22) PCT Filed: Oct. 30, 2008
(86) PCT No.: PCT/AU08/01609

§ 371 Date: Sep. 23, 2010

(30) Foreign Application Priority Data
Oct. 31, 2007 (AU) 2007905977

(51) Int. Cl. A61K 33/06; A61P 43/00

(52) U.S. Cl. 424/682

(57) ABSTRACT

A calcium-rich powder possessing therapeutic properties produced by washing and sterilising the shells of molluscs taken from an uncontaminated marine environment, heating said washed and sterilised shells to a temperature of between 1000° C. and 2000° C. for a period of at least 10 hours, reducing the temperature of the resultant material to room ambient temperature over a period of at least 24 hours, and then crushing said resultant material to a fine powder.

### FIELD OF THE INVENTION

This invention relates to powder having a therapeutic effect and a method of preparing such powder, more specifically relates to a high calcium-containing powder and a method of preparation thereof

### BACKGROUND TO THE INVENTION

With the advancements in science and technology and the development of industries in modern society, the living environment of human beings has been damaged to a serious extent. Due to a number of factors which include serious water and air pollution, pesticide residues on fruit and vegetables, pollution caused by chemicals in western medicines, lack of exercise, greed for food and drink, and a focus on pleasure seeking which brings about pressure and stress, the level of acidic toxins in the human body has been on the increase day by day, which has led to all sorts of diseases occurring at a younger age. For the sake of good health, these chemical acidic toxins must be cleaned up so as to maintain an acid-base balance in the human body. Otherwise, it will be absolutely impossible for these diseases to be cured.

Provision of calcium is one way of maintaining an acid-base balance in the human body. It is known to use egg shells as a mineral and trace element supplying agent for the substitution of calcium. For instance, U.S. Pat. No. 6,344,217 describes a method for the preparation of putamen ovi from egg shell and use of putamen ovi as a medicament. However, temperatures used in the method of preparation and the size of the particles of powder obtained by the method described in the US patent are not sufficiently small to provide the desired effect. Additionally, it is believed that the raw material is not a suitable source of calcium.

### DESCRIPTION OF THE INVENTION

According to one aspect of the present invention there is provided a method for preparing a calcium-rich powder possessing therapeutic properties, said method including the steps of washing and sterilising the shells of molluscs taken from an uncontaminated marine environment, heating said washed and sterilised shells to a temperature of between 1000° C. and 2000° C. for a period of at least 10 hours, reducing the temperature of the resultant material to room ambient temperature over a period of at least 24 hours, followed by crushing of said resultant material to a fine powder.

According to another aspect of the present invention there is provided a calcium-rich powder possessing therapeutic properties produced by washing and sterilising the shells of molluscs taken from an uncontaminated marine environment, heating said washed and sterilised shells to a temperature of between 1000° C. and 2000° C. for a period of at least 10 hours, reducing the temperature of the resultant material to room ambient temperature over a period of at least 24 hours, and then crushing said resultant material to a fine powder.

Preferably the step of washing and sterilising the shells of the molluscs is effected in a strongly alkaline aqueous phase, wherein the strongly alkaline aqueous phase is brought to boiling point, the resultant froth and floating scum is decanted off or ladled off, and the aqueous liquid phase is then poured off the solid shells.

Preferably the temperature at which the washed and sterilized shells are heated is above 1000° C.

Preferably the period of heating of washed and sterilized shells is effected for at least 24 hours, and more preferably for at least 36 hours. Preferably the fine powder after the crushing of the said resultant material is 1000+ mesh.

Preferably the molluscs are selected from land snails, oysters, abalone, or pearl shell.

In addition to calcium, the powder contains zinc, magnesium, potassium, sodium and iron and trace amounts of protein, germanium, selenium and vitamins B1, B2, B6, E, C and D. Typically, the calcium content is greater than 60% by weight. The magnesium content can vary between 0.2% and 0.4% by weight.

The calcium-rich powder of the invention differs from other calcium-rich powders in that the calcium is essentially ionic and while the powder is basic in nature, it differs from ordinary alkaline bases. Excessive intake of ordinary alkali bases may lead to alkali poisoning. The powder must be dissolved or suspended in water before consumption. The aqueous solution or suspension can neutralize the acidic toxins in the human body and maintain an acid-base balance. It is believed that this will improve the human body's natural resistance to disease and enhance the normal functions of the internal organs, normal blood circulation and the regeneration of cells in the human body, and also maintain normal metabolism, endocrine and sexual functions and the health of bones and teeth, and prevent the loss of bone marrow.

The calcium produced by the present invention differs from ordinary calcium. The binding of ordinary calcium may lead to stones in the human body. However, calcium produced according to the present invention can rapidly eliminate acids—the root cause of human stones. This will reduce the size of human stones so that they can be discharged from the body. Therefore it reduces the concerns that people have about such stones.

The powder produced according to the present invention can also be used to rapidly neutralize the toxins in snake and bee venom, pesticides, western medicines, alcohol, nicotine in cigarettes, as well as any chemical acids and toxins in water. It is also believed to be the best beauty product to rapidly remove black spots, freckles, age spots or acne.

An aqueous solution or suspension of the powder produced according to the present invention tastes sweet to a healthy person, astringent to people with gastric conditions, bitter to people with liver problems, and sour to people with heart problems. It has a basic, or salty taste or the smell of urine, preserved duck eggs or fish to people who have kidney, bladder, uterus or ovary problems.

The powder produced according to the invention may be provided in bags or loose in containers. Preferably a small bag of the powder contains 0.2 grams of the powder.

The powder produced according to the invention can be used for a wide range of purposes. For example, one can dissolve or suspend 0.4 grams of the powder in a glass of warm water (300 cc). Drinking 2-4 glasses of such a solution or suspension one after another can immediately kill the pain caused by gastritis, enteritis and gastralgia. All symptoms of nephritis, cystitis, metritis, ovaritis, cholecystitis or inflammation of other internal organs or tissue can be improved by drinking the water prepared with the same amount of powder over a period of around half an hour. The same method can be used to improve the symptoms of other acute conditions such as flu, fever, stroke, cerebral concussion, angina pectoris, heart attack and asthma. Patients with acute appendicitis will have their symptoms alleviated and can smile again after drinking 3-4 glasses of the same dosage in water one after another over a period of around half an hour.

This method can also be used to alleviate the acute pain caused by gallstones or kidney stones blocking the bile or urinary tracts during the discharging process. The patient should keep drinking the suspension or solution until the pain disappears (in around half an hour), which indicates that the stones have been discharged. Any abdominal pain can be alleviated by drinking 1-2 glasses of the suspension or solution in around 10 minutes. Those who suffer food poisoning will be able to smile again after drinking 3-4 glasses of the suspension or solution over a period of around half an hour. Those with only mild food poisoning will be able to go back to work after drinking 2-3 glasses of the suspension or solution in around 15 minutes.

People with pharyngolaryngitis or pharyngalgia may prepare a glass of water (300 cc of cold or warm water) with 0.2 grams of the powder (1 small bag) and hold the suspension or solution in the mouth before swallowing it slowly so as to moisten the affected parts. The pain will be alleviated after doing this several times. People with toothache need to clean the tooth cavities first before rinsing the mouth with water containing the normal dosage of the powder produced according to the present invention. Take a toothpick and wet the tip before dipping it in the powder produced according to the present invention and placing the powder in the cavities. The pain will immediately disappear in around three seconds. Those who suffer toothache but have no cavities may hold the aqueous suspension of the powder produced according to the present invention in their mouths for several minutes before swallowing it. The pain will stop after doing this several times.

Wounds such as knife wounds, abrasions and snake bites may be first cleaned using an aqueous solution or suspension of the powder before some of the powder is directly applied onto the wound. The powder produced according to the present invention will immediately bind and coagulate the blood and stop any bleeding. The coagulated binding will gradually come off after the wound is healed and should not be scratched. There will almost be no scarring left.

For snake bites or wasp stings, it would be best to suck out the venom and drink lots of aqueous suspension or solution prepared with the powder produced according to the present invention. Even if the venom is not sucked out, drinking lots of water prepared with the powder produced according to the present invention will still force the venom out of the body in the form of dark watery blood coming out through the skin.

Treatment of gastric haemorrhage: Mix 1 gram of the powder produced according to the present invention (5 small bags) and half a bowl of flour with water to prepare a paste. Ask the patient to swallow the paste and roll on the floor once or twice so that the paste comes in contact with the affected part. This will alleviate the symptoms.

The powder produced according to the present invention can also be used to treat itchy skin, burns, joint pain, bruises, extravasated blood, swelling, etc.

### DESCRIPTION OF PREFERRED EMBODIMENTS

In order that the invention may be more clearly understood reference is made to preferred embodiments.

A quantity of mollusc shell are taken and cleaned by immersing the shell in a strongly alkaline aqueous solution which is brought to boiling point. The resultant froth and floating scum is removed by decanting or ladling. The still hot liquid is then poured off the solid shells. The cleaned shells are then heated in a furnace or kiln for a period of at least thirty-six (36) hours at a temperature of between 1500° C. and 2000° C. The temperature is then allowed to cool to room ambient temperature over a period of at least twenty-four (24) hours. The resultant material is crushed to a fine powder of 1000+ mesh.

A typical analysis for powder derived from oyster shells by using the above process is as follows:

| | |
|---|---|
| Calcium | 64% |
| Zinc | 0.00046% |
| Magnesium | 0.32% |
| Potassium | 0.034% |
| Sodium | 0.93% |
| Protein | Less than 0.1% |
| Iron | 0.005% |

Typically a powder derived from abalone shells by the above process demonstrates the following analysis:

| | |
|---|---|
| Calcium | 62% |
| Magnesium | 0.21% |
| Sodium | 0.43% |
| Zinc | 0.025% |
| Potassium | 0.022% |
| Protein | 0.04% |

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference to any prior art in this specification is not, and should not be taken as, an acknowledgment or any form or suggestion that the prior art forms part of the common general knowledge in Australia.

Finally, it is to be understood that the foregoing description refers merely to preferred embodiments of the invention, and that variations and modifications will be possible thereto without departing from the spirit and scope of the invention, the ambit of which is to be determined from the following claims.

1. A method for preparing a calcium-rich powder possessing therapeutic properties, said method including the steps of washing and sterilizing the shells of molluscs taken from an uncontaminated marine environment, said step of washing and sterilizing the shells of the molluscs is effected in a strongly alkaline aqueous phase, wherein the aqueous phase is brought to boiling point, the resulting froth and floating scum is decanted off or ladled off, and the aqueous liquid phase is then poured off the solid shells, followed by heating said washed and sterilized shells to a temperature of between 1000° C. and 2000° C. for a period of at least 10 hours, reducing the temperature of the resultant material to room ambient temperature over a period of at least 24 hours, followed by crushing of said resultant material to a fine powder.

2. The method as claimed in claim 1 wherein the said period of heating of said washed and sterilized shells is at least twenty four (24) hours.

3. The method as claimed in claim 2, wherein the period of heating of said washed and sterilized shells is at least thirty six (36) hours.

4. The method as claimed in claim 1 wherein said crushing of said resultant material to a fine powder is carried out to yield a fine powder of 1000+ mesh.

5. The method as claimed in claim 1 wherein said molluscs are selected from oysters, abalone or pearl shell.

6. A calcium-rich powder possessing therapeutic properties produced by washing and sterilizing the shells of molluscs taken from an uncontaminated marine environment, heating said washed and sterilized shells to a temperature of between 1000° C. and 2000° C. for a period of at least 10 hours, reducing the temperature of the resultant material to room ambient temperature over a period of at least 24 hours and then crushing said resultant material to a fine powder.

7. A calcium rich powder as claimed in claim 6, wherein said molluscs are selected from oysters, abalone or pearl shell.

8. A calcium-rich powder as claimed in claim 6 wherein the temperature of heating of said washed and sterilized shells is between 1500° C. and 2000° C.

9. A calcium-rich powder as claimed in claim 6 wherein the period of heating of said washed and sterilized shells is at least twenty four (24) hours.

10. A calcium-rich powder as claimed in claim 9 wherein the period of heating of said washed and sterilized shell is at least thirty six (36) hours.

11. A calcium-rich powder as claimed in claim 6 wherein said powder is at least 1000+ mesh.

12. A method of treatment comprising administration of one or more doses of a solution or suspension of said calcium-rich powder as claimed in claim 6 to a person in need of treatment, each dose of said solution or suspension containing approximately 0.4 grams of said calcium-rich powder in 300 cc of water.

13. A method of treatment comprising administration of said calcium-rich powder as claimed in claim 6 to a person in need of treatment.

14. The method as claimed in claim 2 wherein said molluscs are selected from oysters, abalone or pearl shell.

15. The method as claimed in claim 3 wherein said molluscs are selected from oysters, abalone or pearl shell.

16. The method as claimed in claim 4 wherein said molluscs are selected from oysters, abalone or pearl shell.

17. A calcium-rich powder as claimed in claim 8 wherein the temperature of heating of said washed and sterilized shells is between 1500° C. and 2000° C.

18. A calcium-rich powder as claimed in claim 7 wherein the period of heating of said washed and sterilized shells is at least twenty four (24) hours.

19. A calcium-rich powder as claimed in claim 8 wherein the period of heating of said washed and sterilized shells is at least twenty four (24) hours.

20. A calcium-rich powder as claimed in claim 9 wherein the period of heating of said washed and sterilized shells is at least twenty four (24) hours.

21. A calcium-rich powder as claimed in claim 7 wherein said powder is at least 1000+ mesh.

22. A calcium-rich powder as claimed in claim 8 wherein said powder is at least 1000+ mesh.

23. A calcium-rich powder as claimed in claim 9 wherein said powder is at least 1000+ mesh.

24. A calcium-rich powder as claimed in claim 10 wherein said powder is at least 1000+ mesh.

25. A method of treatment comprising administration of one or more doses of a solution or suspension of said calcium-rich powder as claimed in claim 7 to a person in need of treatment, each dose of said solution or suspension containing approximately 0.4 grams of said calcium-rich powder in 300 cc of water.

26. A method of treatment comprising administration of one or more doses of a solution or suspension of said calcium-rich powder as claimed in claim 8 to a person in need of treatment, each dose of said solution or suspension containing approximately 0.4 grams of said calcium-rich powder in 300 cc of water.

27. A method of treatment comprising administration of one or more doses of a solution or suspension of said calcium-rich powder as claimed in claim 9 to a person in need of treatment, each dose of said solution or suspension containing approximately 0.4 grams of said calcium-rich powder in 300 cc of water.

28. A method of treatment comprising administration of one or more doses of a solution or suspension of said calcium-rich powder as claimed in claim 10 to a person in need of treatment, each dose of said solution or suspension containing approximately 0.4 grams of said calcium-rich powder in 300 cc of water.

29. A method of treatment comprising administration of one or more doses of a solution or suspension of said calcium-rich powder as claimed in claim 11 to a person in need of treatment, each dose of said solution or suspension containing approximately 0.4 grams of said calcium-rich powder in 300 cc of water.

30. A method of treatment comprising administration of said calcium-rich

7. as claimed in claim 7 to a person in need of treatment.

31. A method of treatment comprising administration of said calcium-rich

8. as claimed in claim 8 to a person in need of treatment.

32. A method of treatment comprising administration of said calcium-rich

9. as claimed in claim 9 to a person in need of treatment.

33. A method of treatment comprising administration of said calcium-rich

10. s claimed in claim 10 to a person in need of treatment.

34. A method of treatment comprising administration of said calcium-rich powder as claimed in claim 11 to a person in need of treatment.
